(19) United States
(12) Patent Application Publication
(10) Pub. No.: US 2016/0302652 A1
(43) Pub. Date: Oct. 20, 2016

(54) FLUORESCENCE OBSERVATION APPARATUS

(71) Applicant: OLYMPUS CORPORATION, Tokyo (JP)

(72) Inventor: Koki MORISHITA, Tokyo (JP)

(73) Assignee: OLYMPUS CORPORATION, Tokyo (JP)

(21) Appl. No.: 15/198,407

(22) Filed: Jun. 30, 2016

Related U.S. Application Data
(63) Continuation of application No. PCT/JP2015/050446, filed on Jan. 9, 2015.

(30) Foreign Application Priority Data
Jan. 31, 2014 (JP) 2014-016905

(51) Int. Cl. A61B 1/04; H04N 5/225; G01N 21/64; A61B 1/06; A61B 1/07; G01N 21/47; G02B 23/24; A61B 1/00

(52) U.S. Cl. CPC A61B 1/043; G02B 23/2469; H04N 5/2256; A61B 1/00006; A61B 1/0638; A61B 1/07; G01N 21/474; G01N 21/6456; G02B 2207/113; G01N 2201/08; G01N 2201/061; G01N 2201/0612; G01N 2021/4742

(57) ABSTRACT

A fluorescence observation apparatus including: a light source unit that simultaneously irradiates a subject with illumination light and excitation light having a partial wavelength band of the wavelength band of the illumination light; an objective lens unit that forms an image of reflected light reflected at the subject due to being irradiated with the illumination light and an image of fluorescence generated at the subject due to being irradiated with the excitation light; a single image capturing element that simultaneously acquires the images of reflected light and fluorescence; a filter that is disposed between the objective lens unit and the image capturing and that transmits the light, except the excitation light, to the image capturing element; and a light-adjusting unit that adjusts the output intensity of the illumination light and the output intensity of the excitation light from the light source unit independently of each other.

### CROSS-REFERENCE TO RELATED APPLICATIONS

This is a continuation of International Application PCT/JP2015/050446, with an international filing date of Jan. 9, 2015, which is hereby incorporated by reference herein in its entirety. This application claims the benefit of Japanese Patent Application No. 2014-016905, filed on Jan. 31, 2014, the content of which is incorporated herein by reference.

### TECHNICAL FIELD

The present invention relates to a fluorescence observation apparatus.

### BACKGROUND ART

There is a known fluorescence observation apparatus that uses a single light source and a single image capturing element to simultaneously acquire images of both reflected light of illumination light in the visible range and fluorescence from a subject by the use of a common image capturing element (refer to, for example, Patent Literature PTL 1 below).

In the fluorescence observation apparatus of Patent Literature 1, as the difference in intensity between the fluorescence and the reflected light becomes greater, less intense light is overwhelmed by more intense light, making it difficult to observe the less intense light in the form of an image. If, for example, a reflected-light signal is much more intense than a fluorescence signal, it becomes difficult to clearly observe the fluorescence image.

### CITATION LIST

### Patent Literature

### {PTL 1}

Japanese Unexamined Patent Application, Publication No. 2005-312830

### SUMMARY OF INVENTION

The present invention provides a fluorescence observation apparatus comprising: a light source unit including an illumination light source that emits illumination light and an excitation light source that emits excitation light having a partial wavelength band of the wavelength band of the illumination light, wherein the light source unit simultaneously radiates the illumination light and the excitation light on a subject; an objective lens unit that forms an image of reflected light reflected at the subject due to being irradiated with the illumination light and an image of fluorescence generated at the subject due to being irradiated with the excitation light; single image capturing element that simultaneously acquires the image of reflected light and the image of fluorescence; a filter that is disposed between the objective lens unit and the image capturing element, that cuts off the excitation light, and that transmits all or most of the reflected light except the excitation light; and a light-adjusting unit that adjusts the output intensity of the illumination light from the illumination light source and the output intensity of the excitation light from the excitation light source, independently of each other.

### BRIEF DESCRIPTION OF DRAWINGS

FIG. 1 is an overall structural diagram of a fluorescence observation apparatus according to a first embodiment of the present invention.

FIG. 2 shows graphs illustrating wavelength characteristics of (a) white light, (b) excitation light, (c) output light from a light source unit, and (d) a barrier filter.

FIG. 3 shows graphs illustrating wavelength characteristics of (a) a fluorochrome, (b) fluorescence, (c) reflected light, and (d) incident light on an image capturing element.

FIG. 4 is an overall structural diagram of a fluorescence observation apparatus according to a second embodiment of the present invention.

FIG. 5 is an overall structural diagram showing a modification of the fluorescence observation apparatus in FIG. 4.

FIG. 6 is an overall structural diagram of a fluorescence observation apparatus according to a third embodiment of the present invention.

FIG. 7 shows a graph illustrating wavelength characteristics of three chromatic filters (R, G, and B) provided in a rotating filter of the fluorescence observation apparatus in FIG. 6.

FIG. 8 is a diagram illustrating the operation of the fluorescence observation apparatus in FIG. 6, in the form of graphs showing wavelength characteristics of output light ((a), (c), and (e)) from the light source unit and of incident light ((b), (d), and (f)) on the image capturing element in a first step (a) and (b), a second step (c) and (d), and a third step (e) and (f).

### DESCRIPTION OF EMBODIMENTS

### First Embodiment

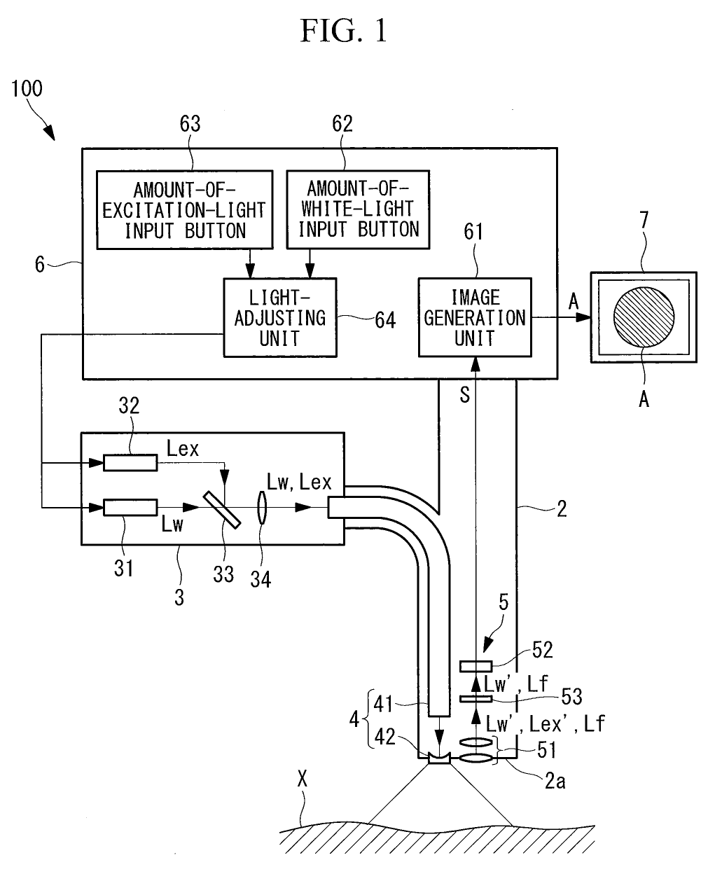

A fluorescence observation apparatus 100 according to a first embodiment of the present invention will now be described with reference to FIGS. 1 to 3.

The fluorescence observation apparatus 100 according to this embodiment is an endoscope apparatus and, as shown in FIG. 1, includes: an elongated insertion section 2 that is inserted into a body; a light source unit 3; an illumination unit 4 that radiates white light (illumination light) Lw and excitation light Lex from the light source unit 3 via a distal end 2a of the insertion section 2 onto biological tissue (subject) X; an image-capturing unit 5 that is provided at the distal end 2a of the insertion section 2 and that acquires image information S of the biological tissue X; an image processor 6 that processes the image information S; and a display unit 7 that displays an image A generated by the image processor 6.

The light source unit 3 includes: a white light source (illumination light source) 31; an excitation light source 32; a dichroic mirror 33 that combines the white light Lw and the excitation light Lex emitted from these two light sources 31 and 32; and a coupling lens 34 that condenses the light combined by the dichroic mirror 33.

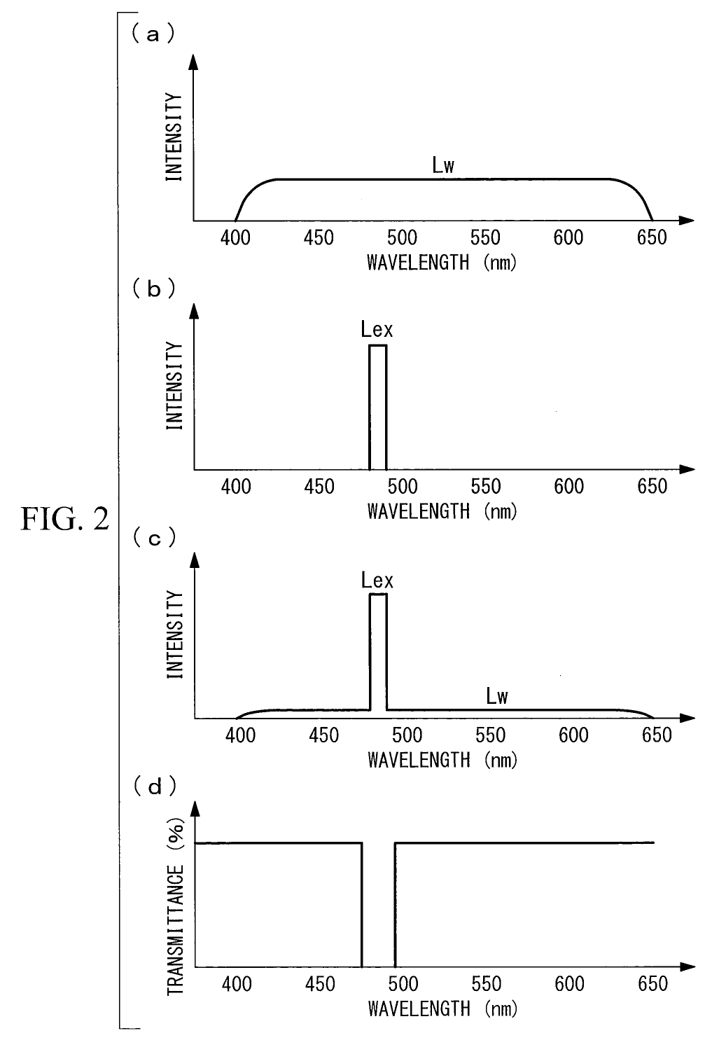

The white light source 31 is a light source employing, for example, a xenon lamp and, as shown in (a) of FIG. 2, emits the white light Lw with wavelengths over the entire visible region (more specifically, over the range from 400 nm to 650 nm).

The excitation light source 32 is a light source employing, for example, a laser diode that emits narrow-band light and, as shown in (b) of FIG. 2, emits the blue excitation light Lex (more specifically, light with wavelengths from 480 nm to 490 nm).

The dichroic mirror 33 reflects the excitation light Lex and transmits the white light Lw, to output light in which the white light Lw and the excitation light Lex are superimposed, as shown in (c) of FIG. 2.

The illumination unit 4 includes a light-guide fiber 41 extending over almost the entire length in the longitudinal direction of the insertion section 2 and an illumination optical system 42 provided at the distal end 2a of the insertion section 2. The light-guide fiber 41 guides the light condenses by the coupling lens 34. The illumination optical system 42 diffuses the white light Lw and the excitation light Lex guided by the light-guide fiber 41, to radiate the light on the biological tissue X opposing the distal end 2a of the insertion section 2.

The image-capturing unit 5 includes an objective lens unit 51 that forms an image of the light from the biological tissue X; an image capturing element 52 that acquires the image formed by the objective lens unit 51; and a barrier filter (filter) 53 disposed between the objective lens unit 51 and the image capturing element 52.

The image capturing element 52 is, for example, a color CCD or a color CMOS and acquires a color image of the light formed by the objective lens unit 51.

As shown in (d) of FIG. 2, the barrier filter 53 has an optical characteristic for blocking light in the wavelength region corresponding to the excitation light Lex and transmitting light in spectral bands other than that wavelength region.

The image processor 6 includes an image generation unit 61 that generates the color image A from the image information S acquired by the image capturing element 52. The image generation unit 61 outputs the generated image A to the display unit 7.

The image processor 6 includes an amount-of-white-light input button 62 and an amount-of-excitation-light input button 63 that can be operated for input by a user and a light-adjusting unit 64 that controls the output intensities of the white light source 31 and the excitation light source 32, independently of each other, according to the inputs to these buttons 62 and 63.

The amount-of-white-light input button 62 and the amount-of-excitation-light input button 63 are provided on the front surface of the image processor 6. The intensity of the white light Lw can be input with the amount-of-white-light input button 62, and the input intensity is transmitted to the light-adjusting unit 64. The intensity of the excitation light Lex can be input with the amount-of-excitation-light input button 63, and the input intensity is transmitted to the light-adjusting unit 64.

The light-adjusting unit 64 adjusts the output intensity of the white light source 31 according to the intensity received from the amount-of-white-light input button 62. The light-adjusting unit 64 adjusts the output intensity of the excitation light source 32 according to the intensity received from the amount-of-excitation-light input button 63.

The operation of the fluorescence observation apparatus 100 having the above-described structure will now be described.

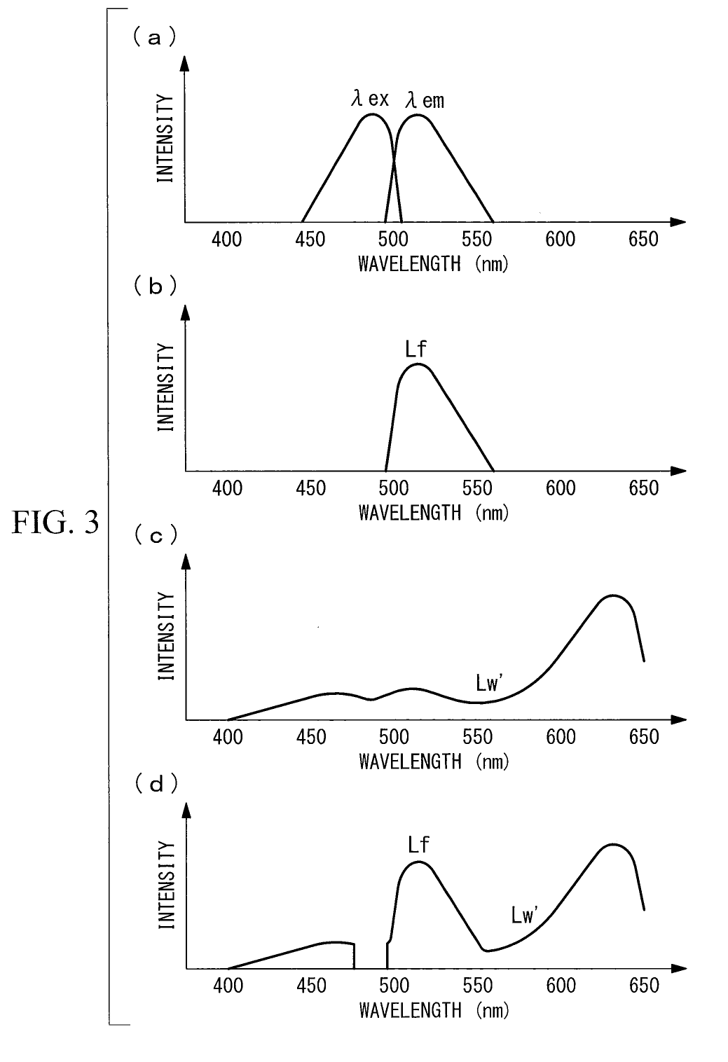

In order to observe the biological tissue X with the fluorescence observation apparatus 100 according to this embodiment, a fluorochrome that accumulates, for example, in a lesion is administered in advance to the biological tissue X. As shown in (a) of FIG. 3, this embodiment assumes a fluorochrome having an excitation wavelength λex of 470 nm to 490 nm and a fluorescence wavelength λem of 510 nm to 530 nm.

First, the insertion section 2 is inserted into the body, then the distal end 2a is disposed so as to face the biological tissue X, and finally the white light Lw and the excitation light Lex are simultaneously radiated via the distal end 2a of the insertion section 2 onto the biological tissue X by the operation of the light source unit 3. In the biological tissue X, reflected light Lw′ (refer to (c) of FIG. 3) is produced as a result of the white light Lw being reflected at the surface of the biological tissue X. At the same time, radiating the excitation light Lex generates two components: fluorescence Lf (refer to (b) of FIG. 3) with wavelengths of 510 nm to 530 nm and reflected light Lex′ of excitation light with wavelengths of 480 to 490 nm.

Some of the reflected light Lw′ and Lex′ of the white light and the excitation light and the fluorescence Lf return to the distal end 2a of the insertion section 2 and are incident upon the objective lens unit 51. Thereafter, the reflected light Lex′ of the excitation light is blocked by the barrier filter 53, and the reflected light Lw′ of the white light and the fluorescence Lf are incident upon the image capturing element 52 (refer to (d) of FIG. 3).

In this manner, images of the reflected light Lw′ and the fluorescence Lf are simultaneously acquired by the common image capturing element 52 for use as the image information S. Next, the image A is generated from the image information S in the image generation unit 61 in the image processor 6, and the generated image A is displayed on the display unit 7. This image A is an image in which the reflected light image and the fluorescence image of the biological tissue X are superimposed.

Here, the brightnesses of the reflected light image and the fluorescence image in the image A are proportional to the respective intensities of the white light Lw and the excitation light Lex radiated onto the biological tissue X. In this embodiment, while observing the image A displayed on the display unit 7, the user can operate the amount-of-white-light input button 62 and the amount-of-excitation-light input button 63 to adjust the output intensity of each of the light sources 31 and 32 independently of each other, thereby adjusting the brightnesses of the reflected light image and the fluorescence image in the image A independently of each other. Therefore, an advantage is afforded in that both the reflected light image and the fluorescence image can be clearly observed by adjusting the output intensity of each of the light sources 31 and 32 with the buttons 62 and 63 so that the reflected light image and the fluorescence image are displayed, for example, with brightnesses substantially identical to each other in the image A.

In this embodiment, it is preferable that the light-adjusting unit 64 set an upper limit for the output intensity of the excitation light source 32 according to the output intensity of the white light source 31.

When intense excitation light Lex is radiated onto the biological tissue X from a near distance, the problem may occur that the biological tissue X is affected by heat or that autofluorescence occurs. On the other hand, always restricting the intensity of the excitation light Lex to a lower value to prevent the above-described problem in case the excitation light Lex is radiated from a near distance may cause the excitation of the fluorochrome to be too insufficient to observe from a far distance.

In this case, the shorter the observation distance (distance between the biological tissue X and the distal end 2a of the insertion section 2), the larger the amount of the reflected light Lw′ incident on the image capturing element 52, and hence the output intensity of the white light source 31 is set to a lower value. Therefore, the biological tissue X can be prevented from being irradiated with intense excitation light Lex from a near distance by setting a lower upper limit for the output intensity of the excitation light source 32 as the output intensity of the white light source 31 becomes lower.

For example, it is assumed that the output intensity of each of the light sources 31 and 32 can be changed in ten levels from “1” through “10”. Here, “1” is the lowest intensity, and “10” is the highest intensity. Even if the output intensity of the white light source 31 and the output intensity of the excitation light source 32 have the same level values, their absolute values differ. For example, even if the level values are the same value “10”, the absolute value of the output intensity of the excitation light source 32 is 100 times as high as the absolute value of the output intensity of the white light source 31.

It is assumed that when the biological tissue X is to be observed from a far distance, the output intensity of the white light source 31 is set to “10” by the user. In this case, the light-adjusting unit 64 sets the upper limit of the output intensity of the excitation light source 32 to “10” and that the output intensity of the excitation light source 32 can be changed in the range from “1” through “10”. Meanwhile, it is assumed that when the biological tissue X is to be observed from a near distance, the output intensity of the white light source 31 is set to “3” by the user. In this case, the light-adjusting unit 64 sets the upper limit of the output intensity of the excitation light source 32 to “3”, and the output intensity of the excitation light source 32 can be changed in the range from “1” through “3”.

In this manner, an upper limit can be set for the output intensity Iex of the excitation light source 32 so that the ratio Iex/Iw of the output intensity Lex of the excitation light source 32 to the output intensity Iw of the white light source 31 is equal to or smaller than a prescribed value, thereby making it possible to adjust the intensity of the excitation light Lex to be radiated onto the biological tissue X within an appropriate range.

### Second Embodiment

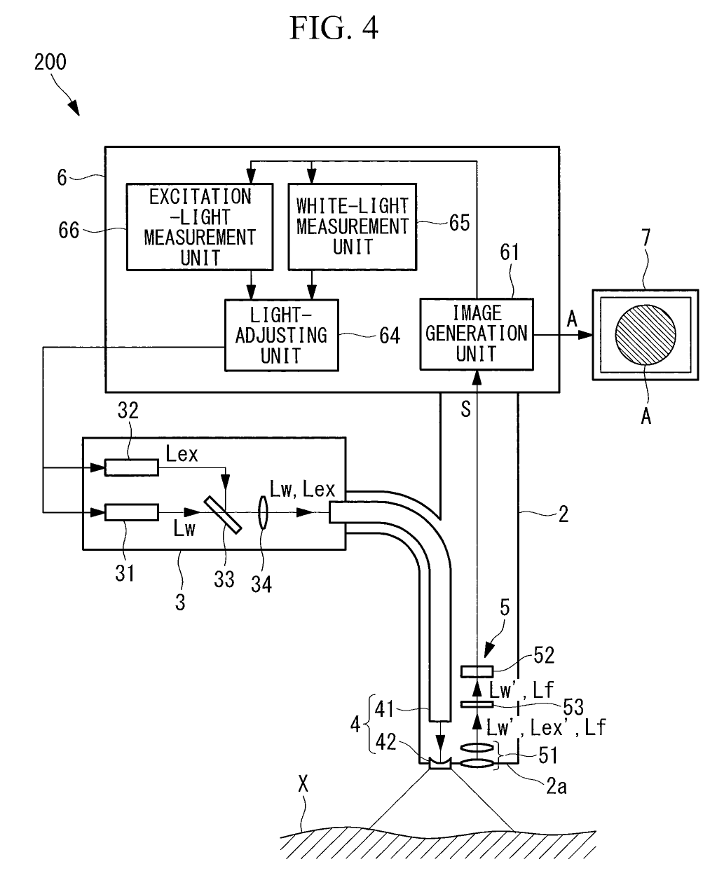

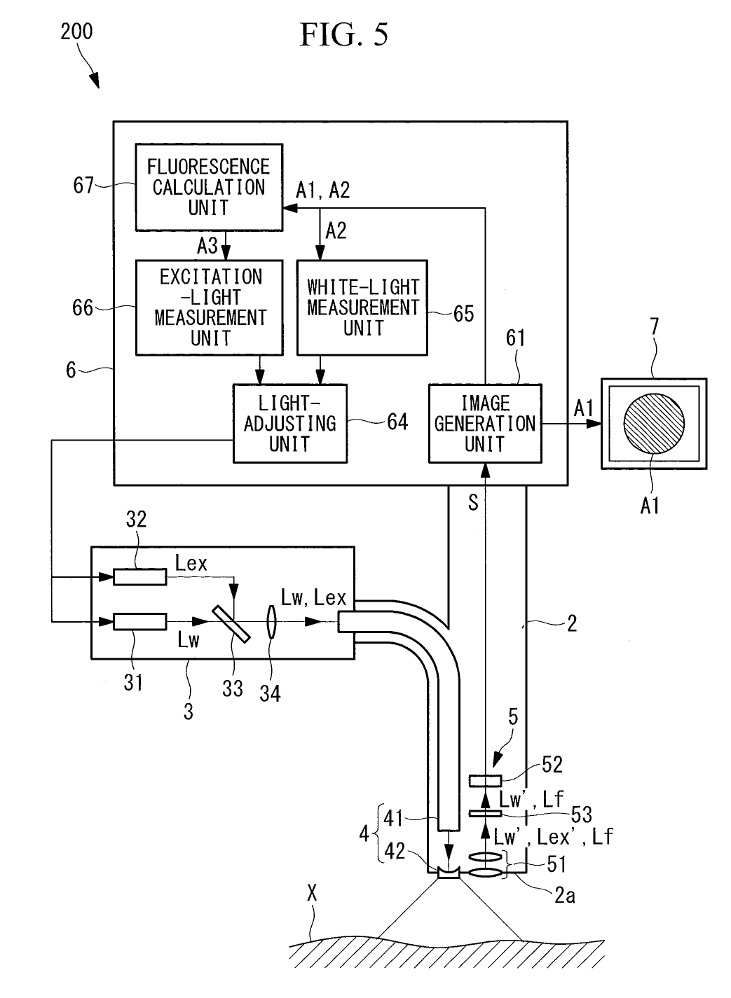

A fluorescence observation apparatus 200 according to a second embodiment of the present invention will now be described with reference to FIGS. 4 and 5.

In this embodiment, differences from the first embodiment will mainly be described, and structures in common with those in the first embodiment will be denoted with the same reference signs, and descriptions thereof will be omitted.

In the first embodiment, the user manually adjusts the brightnesses of the white light Lw and the excitation light Lex radiated on the biological tissue X. This embodiment differs from the first embodiment in that the brightnesses of the white light Lw and excitation light Lex are automatically adjusted.

More specifically, in the fluorescence observation apparatus 200 according to this embodiment, the image processor 6 includes a white-light measurement unit 65 and an excitation-light measurement unit 66, as shown in FIG. 4, instead of the amount-of-white-light input button 62 and the amount-of-excitation-light input button 63.

Of the three monochrome images (i.e., the R image, the G image, and the B image) constituting the color image A, the image generation unit 61 transmits the monochrome image corresponding to the color taken on by the fluorescence Lf to the excitation-light measurement unit 66 and transmits another monochrome image to the white-light measurement unit 65. This embodiment assumes that the G image is transmitted to the excitation-light measurement unit 66 because the fluorescence Lf is green and that the R image is transmitted to the white-light measurement unit 65 because the biological tissue X is a color containing many red components.

The white-light measurement unit 65 calculates a representative value (e.g., mean value or median value) of the brightness values of the R image received from the image generation unit 61 and transmits the obtained representative value to the light-adjusting unit 64. A positive correlation holds between the representative value of the R image and the intensity of the white light Lw. Hence, the white-light measurement unit 65 can measure the intensity of the white light Lw radiated on the biological tissue X from the representative value of the R image.

The excitation-light measurement unit 66 calculates a representative value (e.g., mean value or median value) of the brightness values of the G image received from the image generation unit 61 and transmits the obtained representative value to the light-adjusting unit 64. A positive correlation holds between the representative value of the G image and the intensity of the excitation light Lex. Hence, the excitation-light measurement unit 66 can measure the intensity of the excitation light Lex radiated on the biological tissue X from the representative value of the G image.

The light-adjusting unit 64 controls, on the basis of the representative value received from the white-light measurement unit 65, the output intensity of the white light source 31 so that the representative value becomes equal to a prescribed value. The light-adjusting unit 64 controls, on the basis of the representative value received from the excitation-light measurement unit 66, the output intensity of the excitation light source 32 so that the representative value is within a prescribed value.

The operation of the fluorescence observation apparatus 200 having the above-described structure will now be described.

According to the fluorescence observation apparatus 200 of this embodiment, when the color image A of the biological tissue X is generated in the image generation unit 61, the R image and the G image of the three monochrome images constituting the color image A are transmitted to the white-light measurement unit 65 and the excitation-light measurement unit 66, respectively. Then, in the white-light measurement unit 65, the intensity of the white light Lw radiated on the biological tissue X is measured from the R image brightness, and the white light source 31 is feedback-controlled by the light-adjusting unit 64 so that the intensity of the white light Lw becomes equal to a predetermined value. Meanwhile, in the excitation-light measurement unit 66, the intensity of the excitation light Lex radiated on the biological tissue X is measured from the G image brightness, and the excitation light source 32 is feedback-controlled by the light-adjusting unit 64 so that the intensity of the excitation light Lex becomes equal to a predetermined value.

In this manner, this embodiment affords an advantage in that, by automatically controlling the output intensity of each of the light sources 31 and 32 so that each of the reflected light image and the fluorescence image in the color image A is always displayed at appropriate constant brightness, the user can clearly observe both the reflected light image and the fluorescence image at all times without having to perform a light adjustment operation. Furthermore, when the intensities of the white light Lw and the excitation light Lex radiated on the biological tissue X fluctuate due to, for example, a fluctuation in the observation distance, these intensities are promptly adjusted in an appropriate manner. For this reason, an advantage is afforded in that the biological tissue X can be prevented from being irradiated with white light Lw and excitation light Lex that are more intense than necessary.

The R image is an image of red reflected light, which is only slightly absorbed by the biological tissue X (particularly, blood) and is acquired most stably. By using this R image, an advantage is afforded in that the intensity of the white light Lw radiated on the biological tissue X can be accurately measured and that the output intensity of the white light source 31 can be appropriately controlled. On the other hand, the G image is an image that is only slightly affected by the reflected light Lw′ and that depicts the fluorescence Lf most clearly. By the use of this G image, an advantage is afforded in that the intensity of the excitation light Lex radiated on the biological tissue X can be accurately measured and that the output intensity of the excitation light source 32 can be appropriately controlled.

In this embodiment, as well as in the first embodiment, it is preferable that the light-adjusting unit 64 set an upper limit for the output intensity of the excitation light source 32 according to the output intensity of the white light source 31.

In this embodiment, the white-light measurement unit 65 and the excitation-light measurement unit 66 may calculate the mean value and the maximum value of brightness values of the entirety or part of the color image A, instead of measuring the brightnesses of monochrome images.

If this is the case, the image generation unit 61 transmits, as is, the generated color image A to the white-light measurement unit 65 and the excitation-light measurement unit 66.

The white-light measurement unit 65 calculates the mean value of the brightness values of the entirety or part (preferably middle portion) of the color image A and transmits the obtained mean value to the light-adjusting unit 64.

The excitation-light measurement unit 66 calculates the maximum value of the brightness values of the entirety or part (preferably the middle portion) of the color image A and transmits the obtained maximum value to the light-adjusting unit 64.

The light-adjusting unit 64 controls the output intensity of the white light source 31 so that the received mean value becomes equal to a prescribed value and controls the output intensity of the excitation light source 32 so that the received maximum value becomes equal to a prescribed value.

Because the reflected light image appears in the entire color image A, the effect of a bright local area resulting from the fluorescence Lf can be neglected by the use of the mean value of the brightness values of the entirety or part of the color image A, thereby making it possible to measure the intensity of the white light Lw accurately. On the other hand, because the fluorescence image appears only in a fluorochrome-accumulated local area in the color image A, the intensity of the excitation light Lex can be accurately measured by the use of the maximum value of the brightness values of the color image A.

In this embodiment, the white light source 31 and the excitation light source 32 have been controlled on the basis of the color image A in which the reflected light image and the fluorescence image are superimposed. Instead of this, an image containing only the reflected light image and an image including only the fluorescence image may be generated to control the white light source 31 and the excitation light source 32, respectively, on the basis of these images, as described below.

More specifically, the white light source 31 emits the white light Lw continually, whereas the excitation light source 32 emits the excitation light Lex intermittently by repeatedly turning on/off. This on/off operation of the excitation light source 32 is performed in synchronization with the timing of image acquisition by the image capturing element 52. By doing so, when the excitation light source 32 is on, a first color image A1 in which the fluorescence image and the reflected light image are superimposed is generated from the image information S acquired by the image capturing element 52, and when the excitation light source 32 is off, a second color image A2 containing only the reflected light image is generated from the image information S acquired by the image capturing element 52.

Of the two generated color images A1 and A2, the image generation unit 61 transmits the second color image A2 to the white-light measurement unit 65 and outputs both the color images A1 and A2 to a fluorescence calculation unit 67, as shown in FIG. 5. The fluorescence calculation unit 67 generates a third color image A3 containing only a fluorescence image by subtracting the second color image A2 from the first color image A1 and transmits the obtained third color image A3 to the excitation-light measurement unit 66.

By doing so, the white-light measurement unit 65 can measure the intensity of the white light Lw accurately on the basis of the color image A2 containing only the reflected light image, without being affected by the fluorescence Lf. Furthermore, because the frame rate of the reflected light image does not decrease, the biological tissue X can be finely observed, as usual, on the basis of the reflected light image. On the other hand, the excitation-light measurement unit 66 can measure the intensity of the excitation light Lex accurately on the basis of the third color image A3 containing only the fluorescence image, without being affected by the reflected light Lw′.

### Third Embodiment

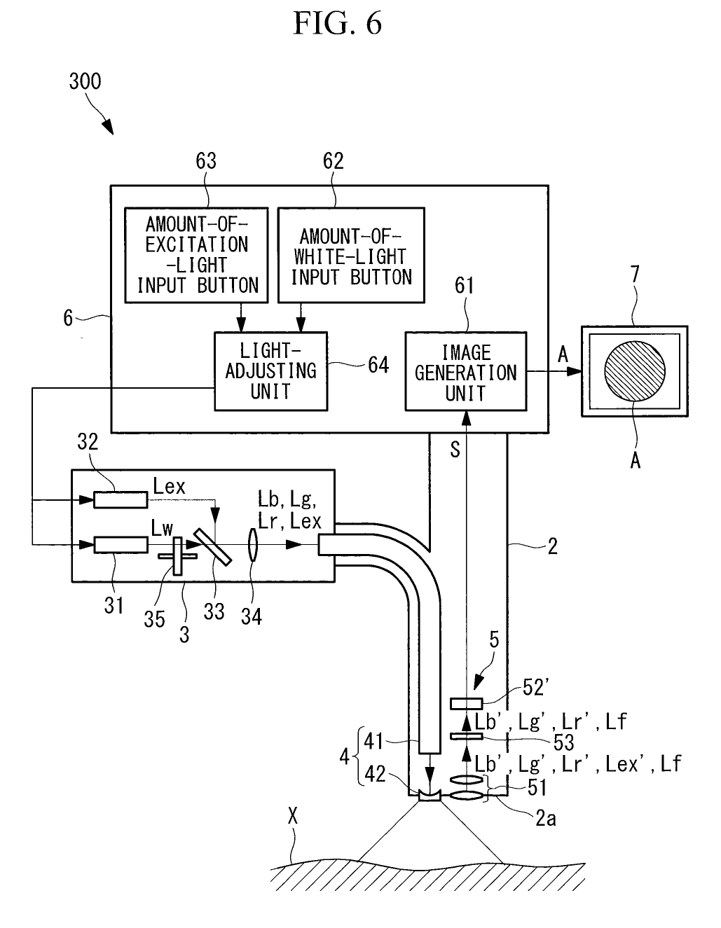

A fluorescence observation apparatus 300 according to a third embodiment of the present invention will now be described with reference to FIGS. 6 to 8.

In this embodiment, differences from the first and second embodiments will mainly be described, and structures in common with those in the first and second embodiments will be denoted with the same reference signs, and descriptions thereof will be omitted.

The first and second embodiments adopt the simultaneous method in which the white light Lw is radiated on the biological tissue X, and an image of that reflected light Lw′ is acquired by the color image capturing element 52. This embodiment differs from the first and second embodiments in that this embodiment employs the frame-sequential method in which blue (B), green (G), and red (R) monochromatic light rays are radiated, in turn, on the biological tissue X, and an image of the reflected light of each of the monochromatic light rays is acquired by a monochrome image capturing element 52′.

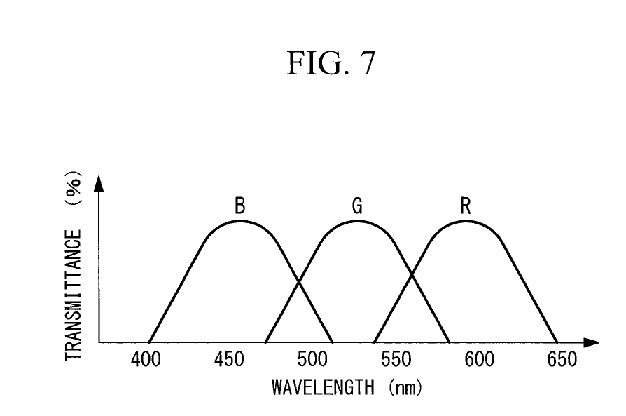

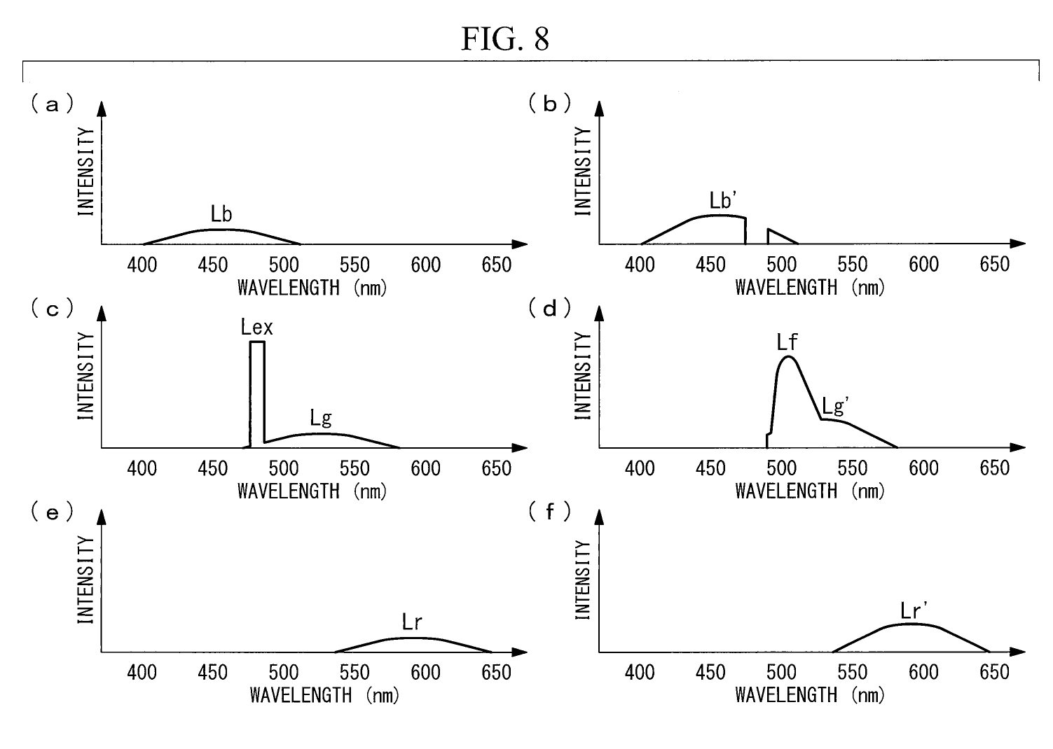

More specifically, the fluorescence observation apparatus 300 according to this embodiment is further provided with a rotating filter 35 between the white light source 31 and the dichroic mirror 33, as shown in FIG. 6. As shown in FIG. 7, the rotating filter 35 includes three types of filters that selectively transmit each of the blue, green, and red light and alternatively positions these three types of filters in turn on the optical path between the white light source 31 and the dichroic mirror 33. By doing so, as shown in (a) to (f) of FIG. 8, the fluorescence observation apparatus 300 acquires, in turn, the B image, the G image, and the R image by repeating a first step to a third step.

More specifically, in the first step, the B image is generated as a result of blue light Lb being radiated on the biological tissue X and an image of reflected light Lb′ of the blue light Lb from the biological tissue X being acquired by the image capturing element 52, as shown in (a) and (b) of FIG. 8. In the second step, the G image is generated as a result of green light Lg being radiated on the biological tissue X and an image of reflected light Lg′ of the green light Lg from the biological tissue X being acquired by the image capturing element 52, as shown in (c) and (d) of FIG. 8. In the third step, the R image is generated as a result of red light Lr being radiated on the biological tissue X and an image of reflected light Lr′ of the red light Lr from the biological tissue X being acquired by the image capturing element 52, as shown in (e) and (f) of FIG. 8. In this case, the excitation light source 32 emits the excitation light Lex in the second step and stops the emission of the excitation light Lex in the first step and the third step. As a result, in the second step, the G image containing a fluorescence image is generated.

The image generation unit 61 combines the three monochrome images into the color image A and outputs the obtained image A to the display unit 7.

When the image capturing elements 52 and 52′ having the same dimensions and the same number of pixels are to be used, the resolution of the image A is generally higher with the frame-sequential method than with the simultaneous method. This is because a monochrome image with a high resolution is obtained. More specifically, the fluorescence observation apparatus 300 according to this embodiment affords an advantage in that the image A with a resolution identical to that in the first and second embodiments can be generated by employing the frame-sequential method, while still using the image capturing element 52′, which is smaller than the image capturing element 52. Other advantages are the same as those of the first and second embodiments, and a description thereof is omitted.

In this embodiment, the white-light measurement unit 65 and the excitation-light measurement unit 66 described in the second embodiment may be included instead of the input buttons 62 and 63. In this case, it is preferable that the white-light measurement unit 65 and the excitation-light measurement unit 66 measure the intensity of each of the light Lw′ and Lf from the R image and the G image.

With the simultaneous method, the fluorescence Lf can be observed not only on the G image but also on the R image. In contrast, with the frame-sequential method, the R image in which the fluorescence Lf is thoroughly excluded is acquired. Therefore, the intensity of the white light Lw can be measured even more accurately by the use of such a R image.

In this embodiment, the excitation light Lex has been radiated on the biological tissue X simultaneously with the green light Lg. Instead of this, the excitation light Lex may be radiated on the biological tissue X simultaneously with the blue light Lb or the red light Lr, and the excitation light Lex may be radiated simultaneously with dichromatic or trichromatic light (namely, in two or more steps of the first step to the third step).

The following invention is derived from the above-described embodiments and modifications thereof.

The present invention provides a fluorescence observation apparatus comprising: a light source unit including an illumination light source that emits illumination light and an excitation light source that emits excitation light having a partial wavelength band of the wavelength band of the illumination light, wherein the light source unit simultaneously radiates the illumination light and the excitation light on a subject; an objective lens unit that forms an image of reflected light reflected at the subject due to being irradiated with the illumination light and an image of fluorescence generated at the subject due to being irradiated with the excitation light; a single image capturing element that simultaneously acquires the image of reflected light and the image of fluorescence; a filter that is disposed between the objective lens unit and the image capturing element, that cuts off the excitation light, and that transmits all or most of the reflected light except the excitation light; and a light-adjusting unit that adjusts the output intensity of the illumination light from the illumination light source and the output intensity of the excitation light from the excitation light source, independently of each other.

According to the present invention, as a result of the illumination light and the excitation light from the light source unit being simultaneously radiated on the subject, reflected light and fluorescence are generated, thereby allowing images of both the reflected light and the fluorescence to be acquired by the common image capturing element. Because of this, both the illumination light image and the fluorescence image of the subject can be simultaneously observed in one image.

In this case, the intensities of the reflected light and the fluorescence occurring at the subject are proportional to the intensities of the illumination light and the excitation light, respectively. Therefore, by adjusting, using the light-adjusting unit, independently of each other, the output intensities of the illumination light source and the excitation light source provided separately, the intensity ratio between the reflected light and the fluorescence is appropriately adjusted so that the signal intensities of the reflected light and the fluorescence become similar to each other, thereby allowing both the reflected light image and the fluorescence image to be clearly and simultaneously observed.

In the above-described invention, the light-adjusting unit may adjust the output intensity of the illumination light source and the output intensity of the excitation light source on the basis of a brightness value of an image of the reflected light and the fluorescence acquired by the image capturing element.

By doing so, the output intensities of the light sources can be automatically adjusted without requiring a user operation.

In the above-described invention, the image acquired by the image capturing element may be a color image, and, of a plurality of monochrome images constituting the color image, the light-adjusting unit may adjust the output intensity of the excitation light source on the basis of a brightness value of a monochrome image corresponding to the color of the fluorescence and may adjust the output intensity of the illumination light source on the basis of a brightness value of another monochrome image.

By doing so, the intensities of the reflected light and the fluorescence can be accurately evaluated on the basis of an image without being affected by each other, allowing the output intensity of each of the light sources to be adjusted more appropriately.

In the above-described invention, the light-adjusting unit may adjust the output intensity of the illumination light source on the basis of a mean value of a brightness value of the entirety or part of the image and may adjust the output intensity of the excitation light source on the basis of a maximum value of a brightness value of the entirety or part of the image.

By doing so, the intensity of the reflected light occurring over a wide range on the subject can be evaluated more accurately with the mean value of brightness value of the image. On the other hand, the intensity of the fluorescence occurring at a local area on the subject can be evaluated more accurately with the maximum value of brightness values of the image.

In the above-described invention, the light source unit may continuously radiate the illumination light on the subject and intermittently radiate the excitation light on the subject, wherein the image capturing element may acquire a first image while both the excitation light and the illumination light are being radiated on the subject and may acquire a second image while only the illumination light is being radiated on the subject, and the light-adjusting unit may adjust the output intensity of the illumination light source on the basis of a brightness value of the second image and may adjust the output intensity of the excitation light source on the basis of a brightness values of a third image obtained by subtracting the second image from the first image.

By doing so, the intensity of the reflected light can be evaluated more accurately by using the second image containing only the reflected light image. On the other hand, the intensity of the fluorescence can be evaluated more accurately by using the third image containing only the fluorescence image.

### REFERENCE SIGNS LIST

100, 200, 300 Fluorescence observation apparatus
2 Insertion section
3 Light source unit
31 White light source (illumination light source)
32 Excitation light source
33 Dichroic mirror
34 Coupling lens
35 Rotating filter
4 Illumination unit
41 Light-guide fiber
42 Illumination optical system
5 Imaging unit
51 Objective lens unit
52, 52′ Image capturing element
53 Barrier filter
6 Image processor
61 Image generation unit
62 Amount-of-white-light input button
63 Amount-of-excitation-light input button
64 Light-adjusting unit
65 White-light measurement unit
66 Excitation-light measurement unit
67 Fluorescence calculation unit
X Biological tissue (subject)
Lw White light (illumination light)
Lw′ Reflected light
Lex Excitation light
Lf Fluorescence

1. A fluorescence observation apparatus comprising:
a light source unit including an illumination light source that emits illumination light and an excitation light source that emits excitation light having a partial wavelength band of the wavelength band of the illumination light, wherein the light source unit simultaneously radiates the illumination light and the excitation light on a subject;
an objective lens unit that forms an image of reflected light reflected at the subject due to being irradiated with the illumination light and an image of fluorescence generated at the subject due to being irradiated with the excitation light;
a single image capturing element that simultaneously acquires the image of reflected light and the image of fluorescence;
a filter that is disposed between the objective lens unit and the image capturing element, that cuts the excitation light, and that transmits all or most of the reflected light except the excitation light; and
a light-adjusting unit that adjusts the output intensity of the illumination light from the illumination light source and the output intensity of the excitation light from the excitation light source, independently of each other.

2. The fluorescence observation apparatus according to claim 1, wherein the light-adjusting unit adjusts the output intensity of the illumination light source and the output intensity of the excitation light source on the basis of a brightness value of an image of the reflected light and the fluorescence acquired by the image capturing element.

3. The fluorescence observation apparatus according to claim 2, wherein the image acquired by the image capturing element is a color image, and
of a plurality of monochrome images constituting the color image, the light-adjusting unit adjusts the output intensity of the excitation light source on the basis of a brightness value of a monochrome image corresponding to the color of the fluorescence and adjusts the output intensity of the illumination light source on the basis of a brightness value of another monochrome image.

4. The fluorescence observation apparatus according to claim 2, wherein the light-adjusting unit adjusts the output intensity of the illumination light source on the basis of a mean value of a brightness value of the entirety or part of the image and adjusts the output intensity of the excitation light source on the basis of a maximum value of a brightness value of the entirety or part of the image.

5. The fluorescence observation apparatus according to claim 2, wherein the light source unit continuously radiates the illumination light on the subject and intermittently radiates the excitation light on the subject,
wherein the image capturing element acquires a first image while both the excitation light and the illumination light are being radiated on the subject and acquires a second image while only the illumination light is being radiated on the subject, and
wherein the light-adjusting unit adjusts the output intensity of the illumination light source on the basis of a brightness value of the second image and adjusts the output intensity of the excitation light source on the basis of a brightness value of a third image obtained by subtracting the second image from the first image.
